(19) United States
(12) Patent Application Publication
(10) Pub. No.: US 2006/0060202 A1
(43) Pub. Date: Mar. 23, 2006

(54) Gastric tube placement indicator

(76) Inventors: Daniel P. Flynn, Florissant, MO (US); Glenn G. Fournie, Smithton, IL (US); Kevin C. Meier, Affton, MO (US); Paul Trelford, St. Louis, MO (US)

Correspondence Address: Tyco Healthcare Group, LP;Intellectual Property Department Docketing, 15 Hampshire Drive, Mansfield, MA, 02048, US

(21) Appl. No.: 11/139,118

(22) Filed: May 27, 2005

Related U.S. Application Data
(63) Continuation-in-part of application No. 10/945,758, filed on Sep. 21, 2004, now abandoned.

(51) Int. Cl. A62B 9/06; A61M 16/00

(52) U.S. Cl. 128/207.14; 128/207.15

(57) ABSTRACT

A gastric tube placement device having a carbon dioxide indicator is disclosed. The carbon dioxide indicator comprises a rectangular housing defining a chamber in communication with opposing ports that permits substantial axial flow through the chamber with a carbon dioxide detector disposed therein. A Y-port connector having first and second legs in communication with a main port with a gastric tube coupled to the inside of the connector is provided for insertion of the distal end of the gastric tube through the esophagus of the patient. The carbon dioxide indicator further comprises a carbon dioxide detector disposed inside the rectangular housing which is configured to minimize the dead space inside the housing and facilitates a substantially axial airflow through the carbon dioxide detector when a syringe or similar air-evacuating device is engaged to the rectangular housing and draws air from the housing.

### CROSS-REFERENCE TO RELATED APPLICATION

This continuation-in-part application claims the benefit of U.S. Non-Provisional Patent Application entitled “Gastric Tube Placement Indicator”, Ser. No. 10/945,758, filed Sep. 21, 2004, which is herein incorporated by reference.

### FIELD OF THE INVENTION

The present invention relates to a medical device employed to verify placement of a gastric feeding tube in a patient, and more particularly to a gastric tube placement device for the detection of carbon dioxide through a gastric feeding tube.

### BACKGROUND OF THE INVENTION

It is known in the art that gastric feeding tubes may be employed for feeding patients requiring nutritional support. Such gastric tubes can be inserted into a patient either orally or nasally. In practice, a gastric feeding tube is inserted either into the mouth or nose of the patient and through the patient's pharynx until it reaches the esophagus.

A common drawback when placing gastric feeding tubes either orally or nasally is the potential of passing the gastric feeding tube into the trachea, and then deeper into the respiratory tract and lungs, instead of properly in the stomach. The consequence of having a gastric feeding tube placed into the respiratory system can lead to adverse medical complications, including pneumothorax, aspiration pneumonia or other complications that can damage the patient's respiratory system.

Accordingly, methods for confirming the proper placement of the gastric feeding tube in the esophagus have been developed, such as fluoroscopy, chest X-rays, and continuous carbon dioxide monitoring (i.e., capnography). However, fluoroscopy and chest X-rays are disadvantageously time consuming, relatively expensive, and can expose the patient to high doses of radiation, while carbon dioxide detection machines used in capnography are relatively expensive and complex compared to other means of monitoring carbon dioxide.

Colorimetric carbon dioxide detectors have been commonly used with ventilator systems for detecting the presence of carbon dioxide for proper placement of a tracheal tube into the trachea of a patient. The calorimetric indicator has a pH sensitive paper that changes color in the presence of carbon dioxide for visually indicating to the healthcare practitioner that the trachea tube is properly placed into the trachea, rather than the esophagus. Although such calorimetric indicators adequately detect the presence of carbon dioxide in the respiratory system during placement of the trachea tube, the use of conventional calorimetric indicators for use in indicating improper placement of the gastric feeding tube in the trachea is disadvantageous. Because the lumen of a gastric tube is much smaller than the larger lumen of a trachea tube the capacity for facilitating sufficient airflow for the quick detection of carbon dioxide through the smaller lumen gastric feeding tube is limited.

Referring to FIG. 1, the housing 88 of the prior art calorimetric carbon dioxide indicator 8 may comprise inlet and outlet ports 90 and 92 positioned in perpendicular relationship to one another relative to housing 88. In addition, housing 88 of the carbon dioxide indicator 8 defines a necessarily large volume since the inlet and outlet ports 90 and 92 are required to be sized and shaped to engage the relatively large lumen of the ventilation tubing associated with a ventilation system in comparison with the relatively smaller lumen of the gastric feeding tube used for feeding applications. The larger ports 90 and 92 of the prior art carbon dioxide indicator 8 also increases the size and volume of the indicator housing 88 to accommodate these ports 90 and 92 which necessarily increases the potential dead space defined by housing 88. As such, the use of a prior art carbon dioxide indicator 8 for gastric tube placement is problematic since the gastric feeding tube has a relatively smaller lumen than a trachea tube for respiratory applications that can create insufficient airflow through the larger dead space defined by the housing 88 for quick detection of carbon dioxide. For example, the housing 88 of a prior art carbon dioxide indicator 8 can have a volume of 5 cubic centimeters with the inlet and outlet ports 90 and 92 that are positioned perpendicular to one another as noted above to accommodate ventilation tubing. Although such prior art carbon dioxide indicators 8 are appropriate for respiratory applications, the larger volume of the indicator 8 and the perpendicular relationship of the outlet and inlet ports 90 and 92 make such indicators 8 unsuitable for gastric tube placement applications because the larger dead space and perpendicular airflow pathway defined between the ports 90 and 92 can decrease the effectiveness of the carbon dioxide indicator 8 to quickly detect the presence of carbon dioxide. In particular, the positioning of such ports creates a perpendicular air flow pathway through the housing of the prior art carbon dioxide detector which is undesirable for gastric tube placement where the emphasis for quickly detecting the presence of carbon dioxide is critical.

Therefore, there is a need in the art for a carbon dioxide indicator for gastric feeding tube placement having a housing that defines a sufficiently low dead space and provides a direct airflow pathway between the inlet and outlet ports.

### SUMMARY OF THE INVENTION

In one embodiment, the present invention comprises a medical placement indicator comprising a rectangular housing, the rectangular housing defining a passageway in communication with opposing first and second ports, the rectangular housing further including a transparent portion for viewing said passageway, and a carbon dioxide detector axially disposed within the passageway, the carbon dioxide detector being adapted to detect the presence of carbon dioxide, the rectangular housing configured to define a low dead space within the rectangular housing, wherein the opposing first and second ports communicate with the passageway such that airflow through the passageway enters through the opposing first port and exits out the opposing second port, and wherein the airflow is directed substantially axial through the passageway of the rectangular housing between the opposing first and second ports.

In another embodiment, the present invention comprises a gastric tube placement device comprising a gastric tube defining a lumen in communication with a distal opening and a proximal opening, and a carbon dioxide indicator including a carbon dioxide detector disposed inside a rectangular housing, the rectangular housing defining a passageway in communication with opposing first and second ports with the carbon dioxide detector being disposed across the passageway, the rectangular housing being configured to define a low dead space within the passageway when the carbon dioxide detector is disposed within the passageway, one of the opposing first and second ports being adapted for engagement with the gastric tube for establishing fluid flow communication between the distal opening of the gastric tube and the passageway of the rectangular housing.

In a further embodiment, a method for detecting gastric tube placement comprises providing a hollow Y-port connector defining first and second legs in communication with a main port; engaging a carbon dioxide indicator comprising a rectangular housing to one of the first and second legs, the rectangular housing defining a passageway in communication with opposing first and second ports, the rectangular housing further including a transparent portion for viewing said passageway, and a carbon dioxide detector axially disposed within said passageway, the carbon dioxide detector being adapted to detect the presence of carbon dioxide, the rectangular housing configured to define a low dead space within the rectangular housing; establishing fluid flow communication between one of the opposing first and second ports with one of the first and second legs; engaging a gastric tube to the main port of the Y-port connector; engaging a means for evacuating air to the rectangular housing; and evacuating air from the rectangular housing such that a substantially axial airflow is initiated through the passageway between the opposing first and second ports such that the carbon dioxide indicator may detect the presence of carbon dioxide in the airflow.

### BRIEF DESCRIPTION OF THE DRAWINGS

FIG. 1 is a perspective view of a prior art carbon dioxide indicator;

FIG. 2 is a perspective view of the carbon dioxide indicator according to the present invention;

FIG. 3 is a top view of the carbon dioxide indicator according to the present invention;

FIG. 4 is a side view of the carbon dioxide indicator according to the present invention;

FIG. 5 is a cross-sectional view taken along line 5-5 of FIG. 4 illustrating the airflow pathway through the carbon dioxide indicator according to the present invention;

FIG. 6 is an exploded view of the carbon dioxide indicator showing the carbon dioxide detector according to the present invention;

FIG. 7 is top partial cross-sectional view of a gastric tube placement device including the carbon dioxide indicator according to the present invention; and

FIG. 8 is an illustration showing the gastric tube placement device being inserted into the esophagus of a patient according to the present invention.

Corresponding reference characters indicate corresponding elements among the view of the drawings.

### DETAILED DESCRIPTION OF THE INVENTION

Referring to the drawings, a gastric tube placement device according to the present invention is illustrated and generally indicated as 10 in FIGS. 2-8. The gastric tube placement device 10 comprises a carbon dioxide (CO2) indicator 12 that encases a CO2 detector 17 in communication with a conventional Y-port connector 16 engaged to a gastric tube 14 for detecting the presence of carbon dioxide from a patient.

Referring to FIGS. 2-4, the CO2 indicator 12 comprises a rectangular housing 18 that encases the CO2 detector 17 for the detection of carbon dioxide that may enter the detector 17 when the gastric tube 14 is placed inside the patient. The housing 18 consists of a lower housing 20 engaged to an upper housing 22 that collectively defines a passageway 44 adapted to receive the CO2 detector 12 axially disposed therein. The housing 18 includes opposing first and second ports 30 and 32 wherein first port 30 is in communication with a barbed connector 34 for connection to the Y-port connector 16 and second port 32 is in communication with a tubular connector 36 adapted to engage a syringe 50 (FIG. 8) or similar air-evacuating device for evacuating air through passageway 44, such as a bellows or flexible bulb, as shall be discussed in greater detail below.

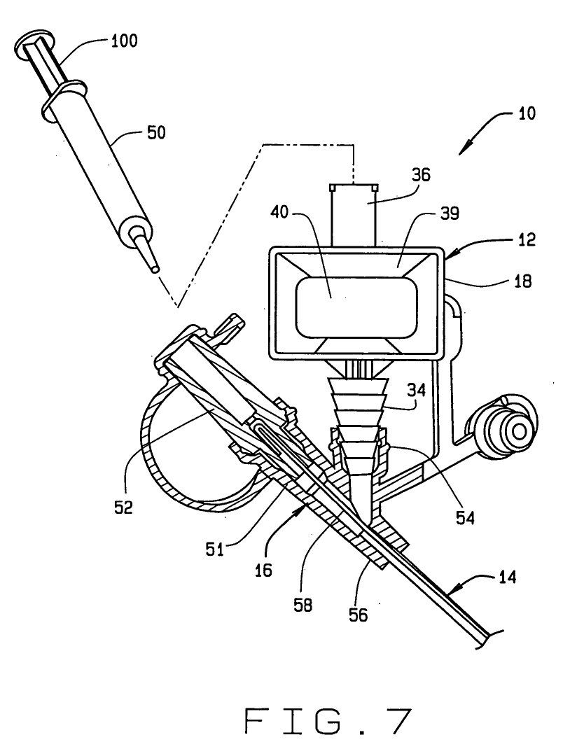

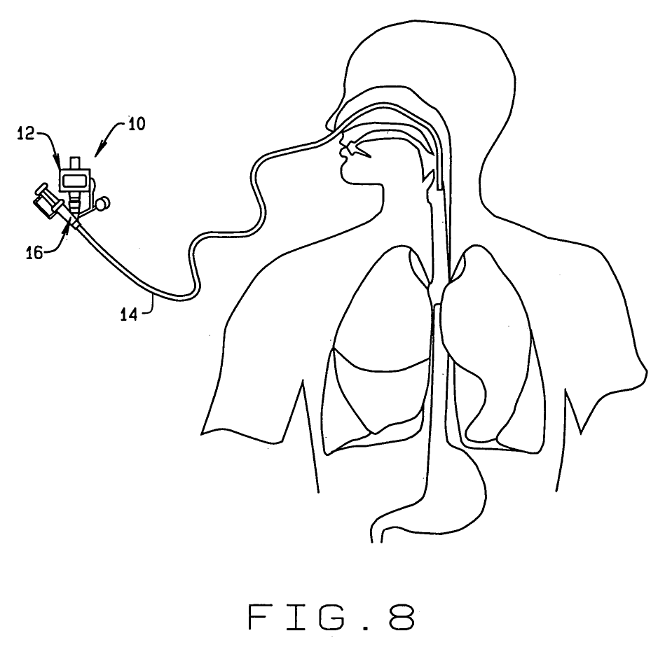

Referring to FIG. 7, the Y-port connector 16 comprises a hollow body 51 defining a first leg 52 and a second leg 54 in communication with a main port 56. The gastric tube 14 is anchored inside the body 51 through the main port 56 such that airflow from the proximal end of the gastric tube 14 communicates with the second leg 54. In assembly, the barbed connector 36 of CO2 indicator 12 is engaged to the second leg 54 of the Y-port connector 16 such that the airflow from the gastric tube 14 communicates with the passageway 44 defined by housing 18.

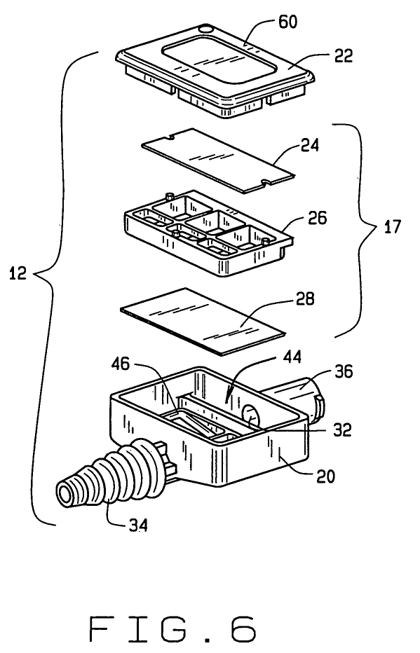

Referring to FIG. 6, the CO2 detector 17 comprises a detector element 24, preferably a calorimetric paper, having a pH sensitive chemical compound that is suspended in a suitable dye in order to undergo a color change as a result of a change in the pH of the calorimetric paper caused by the influx of carbon dioxide carried in a patient's breath when the distal end of the gastric tube 14 is placed in the respiratory tract of the patient. The lower housing 20 defines a filter support 46 that supports a filter 28 that provides a means for filtering the airflow of any contaminants or fluids. Preferably, the filter 28 is fabricated from polypropylene.

In addition, the detector element 24 is carried by a baffled element support 26 positioned above the filter 28 that permits airflow to contact the detector element 24 as air passes through the passageway 44. The CO2 detector 17 is configured such that airflow 42 through the passageway 44 and the detector 17 is substantially axial between the opposing first and second ports 30 and 32 as illustrated in FIG. 5.

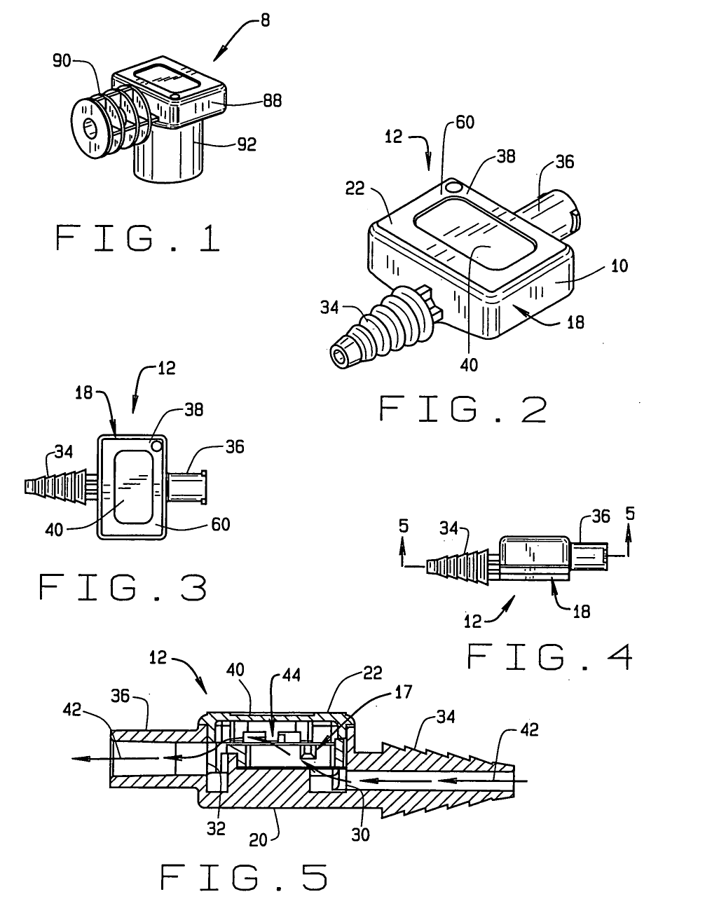

The present invention contemplates that the housing 18 is configured to minimize dead space in passageway 44 when the CO2 detector 17 is disposed axially therein. Preferably, the housing 18 has a volume of 2 cubic centimeters compared to a volume of 5 cubic centimeters for the prior art carbon dioxide indicator shown in FIG. 1. As such, airflow 42 through chamber 44 takes a substantially axial pathway between the opposing first and second ports 30 and 32 that optimizes the exposure of the detector element 24 to carbon dioxide since such airflow 42 takes a substantially axial pathway between the opposing first and second ports 30 and 32 with minimal dead space to divert such airflow. This optimization of exposing the detector element 24 to carbon dioxide entrained in the axial airflow 42 in combination with the minimal dead space and smaller volume of the housing 18 provides a means for allowing the detector element 24 to quickly indicate the presence of carbon dioxide.

As further shown, the upper housing 22 comprises a transparent portion 40 having a graduation display 38 along the peripheral portion thereof having a color scheme for determining whether the color displayed by the CO2 detector 17 through the transparent portion 40 indicates the presence or absence of carbon dioxide by the detector element 24. Preferably, the graduation display 38 includes a color coded chart 60 that comprises a color range that is compared against the color change in the colorimetric paper of the detector element 24 in order to determine the presence of carbon dioxide. Most preferably, the color range includes a yellow color that indicates the presence of carbon dioxide while a purple color indicates that carbon dioxide is not present. Although the detector element 24 of the present invention indicates the presence of carbon dioxide, the detector element 24 does not provide a measurement of the amount of carbon dioxide present since the CO2 indicator 12 lacks any type of means for measuring the degree of carbon dioxide.

During the gastric tube placement procedure, the distal end of the gastric tube 14 is inserted through either the patient's nasal or oral cavity. If a small bore gastric tube 14 is used, a guide wire (not shown) may be disposed inside the lumen of the gastric tube 14 in order to facilitate advancement of the tube 14 into the esophagus of the patient, while use of a large bore gastric tube 14 does not require the use of such a guide wire. To assemble, the barbed connector 34 of the CO2 indicator 12 is attached to the second leg 54 of the Y-port connector 16 and a syringe 50 is attached to the tubular connector 36 in order to obtain a reading as the gastric tube 14 is inserted through the patient's pharynx. During insertion of the gastric tube 14, the user actuates the syringe 50 by pulling back on a plunger 100 such that airflow 42 is established through CO2 indicator 12 as illustrated in FIG. 5. This action of establishing airflow 42 in combination with the minimal volume and dead space defined by housing 18 further enhances the capability of the CO2 indicator 12 to detect the presence of carbon dioxide through gastric tube 14.

In order to ensure that the distal end of the gastric tube 14 passes through the patient's esophagus, rather than the trachea, the user views the detector element 24 through the transparent portion 40 for indicating the presence of carbon dioxide. If the distal end of gastric tube 14 passes into the trachea, the presence of carbon dioxide in sufficient quantity will be detected by the detector element 24 as the calorimetric paper changes to a yellow color, thereby signaling the user that the distal end of the gastric tube 14 has been improperly positioned in the patient's respiratory system. The gastric tube 14 may then be partially withdrawn and reinserted until the distal end of the gastric tube 14 passes by the trachea opening and into the patient's esophagus. Such placement of the gastric tube 14 will indicate little or no carbon dioxide adjacent the distal end of the gastric tube 14.

Once the gastric tube 14 has been properly placed with the distal end of the gastric tube 14 in the patient's esophagus and in communication with the patient's stomach, the gastric tube 14 may then be advanced, if desired, to the small intestine where the guide wire can then be removed when utilized. The patient may then be fed by the normal technique of passing liquid food through the first leg 52 of the Y-port connector 16 for delivery to the small intestine through the gastric tube 14.

It should be understood from the foregoing that, while particular embodiments of the invention have been illustrated and described, various modifications can be made thereto without departing from the spirit and scope of the invention as will be apparent to those skilled in the art. Such changes and modifications are within the scope and teaching of this invention as defined in the claims appended hereto.

1. A medical placement indicator comprising:
a rectangular housing, said rectangular housing defining a passageway in communication with opposing first and second ports, said rectangular housing further including a transparent portion for viewing said passageway, and
a carbon dioxide detector axially disposed within said passageway, said carbon dioxide detector being adapted to detect the presence of carbon dioxide, said rectangular housing being configured to define a low dead space within said rectangular housing, wherein said opposing first and second ports communicate with said passageway such that airflow through said passageway enters through said opposing first port and exits out said opposing second port, and wherein said airflow is directed substantially axial through said passageway of said rectangular housing between said opposing first and second ports.

2. The medical placement indicator according to claim 1, wherein said carbon dioxide detector comprises a detector element adapted to change color in the presence of carbon dioxide.

3. The medical placement indicator according to claim 1, wherein said carbon dioxide detector further comprises an element support engaged to engage the detector element.

4. The medical placement indicator according to claim 1, wherein said first opposing port is integral to said rectangular housing and adapted to engage a leg of a Y-port connector.

5. The medical placement indicator according to claim 4, wherein said first opposing port is a barbed connector.

6. The medical placement indicator according to claim 1, wherein said second opposing port defines a tubular member adapted to operatively engage a means for evacuating air from said rectangular housing.

7. The medical placement indicator according to claim 6, wherein said means for evacuating air comprises a bellows.

8. The medical placement indicator according to claim 6, wherein said means for evacuating air comprises a syringe.

9. A gastric tube placement device comprising:
a gastric tube defining a lumen in communication with a distal opening and proximal opening, and
a carbon dioxide detector disposed inside said rectangular housing, said housing defining passageway in communication with opposing first and second ports with said carbon dioxide detector disposed across said passageway, said carbon dioxide detector being configured to define a low dead space within said passageway, one of said opposing first and second opposing ports adapted for engagement with said gastric tube for establishing fluid flow communication between said distal opening of said gastric tube and said passageway of said rectangular housing, wherein a substantially axial airflow is established between said opposing first and second ports.

10. The gastric tube placement device according to claim 9, wherein another of said opposing first and second ports adapted for engagement to a means for evacuating air from said rectangular housing.

11. A method for detecting gastric tube placement comprising:
a) providing a hollow Y-port connector defining first and second legs in communication with a main port said main port;
b) engaging a carbon dioxide indicator comprising rectangular housing to one of said first and second legs, said rectangular housing defining a passageway in communication with opposing first and second opposing ports, said rectangular housing further including a transparent portion for viewing said passageway, and a carbon dioxide detector disposed across said passageway, said carbon dioxide detector being adapted to detect the presence of carbon dioxide and configured to define a minimal dead space within said passageway;
c) establishing fluid flow communication between one of said opposing first and second ports with one of said first and second legs;
d) engaging a gastric tube to said main port of said Y-port connector;
e) engaging a means for evacuating air to said rectangular housing; and
f) evacuating air from said rectangular housing such that substantially axial airflow is initiated though said passageway between said opposing first and second ports such that said carbon dioxide detector may detect the presence of any carbon dioxide in said airflow.

12. The method according to claim 11, further including the step of inserting a guide wire through another of said legs for facilitating insertion of said gastric tube into the esophagus of a patient.

13. The method according to claim 12, wherein said guide wire is operatively engaged to said gastric tube.
